(19) United States
(12) Patent Application Publication
(10) Pub. No.: US 2006/0171988 A1
(43) Pub. Date: Aug. 3, 2006

(54) Method of treatment using foams as artificial lymph nodes

(76) Inventors: Norbert Hilf, Kirchentellensfurt (DE); Juergen Pinocy, Stuttgart (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/045,596

(22) Filed: Jan. 28, 2005

(51) Int. Cl. A61K 39/00; A61K 39/38; A61F 2/00; A61K 38/17

(52) U.S. Cl. 424/426; 424/184.1; 424/277.1; 514/12

(57) ABSTRACT

The present invention relates to pharmaceutical compositions for the improved activation of cells of the immune system that comprise a biocompatible implantable and an active pharmaceutical ingredient to be integrated into said foam, wherein said composition specifically activates cells of the immune system. In one preferred embodiment, said active pharmaceutical ingredient is selected from endogenous antigens that are selected from tumor-associated antigens. Said foam material can be implanted into a subject in order to produce the pharmaceutical composition in situ. Preferably, said pharmaceutical composition can be present in the form of an artificial lymph node, wherein said artificial lymph node functions as a reservoir for cells of the immune system. The invention also relates to methods for improved immunotherapy, in particular cancer immunotherapy.

The present invention relates to pharmaceutical compositions for the improved activation of cells of the immune system that comprise a biocompatible implantable and an active pharmaceutical ingredient to be integrated into said foam, wherein said composition specifically activates cells of the immune system. In one preferred embodiment, said active pharmaceutical ingredient is selected from endogenous antigens that are selected from tumor-associated antigens. Said foam material can be implanted into a subject in order to produce the pharmaceutical composition in situ. Preferably, said pharmaceutical composition can be present in the form of an artificial lymph node, wherein said artificial lymph node functions as a reservoir for cells of the immune system. The invention also relates to methods for improved immunotherapy, in particular cancer immunotherapy.

### BACKGROUND OF THE INVENTION

The mammalian immune system provides a mechanism of defense against both foreign pathogens like bacteria, eukaryotic parasites, toxins or viruses, and altered cells of the mammalian body, like benign or malignant neoplasia. The recognition of foreign or self substances by the immune system of mammals, e.g., rodents, ruminants, and, in particular humans is mediated by the immune system and in particular the components of the immune system that are, for example, phagocytic cells, natural killer cells, the complement system, B-cell-receptors, various classes of highly specific antibodies produced by plasma-B-cells, and T lymphocytes.

T lymphocytes with specific T cell receptors fall into two major classes: helper T cells, which are positive for the CD4 co-receptor, and killer T cells, which are positive for the CD8 co-receptor. T cell receptors on the cell surfaces of these populations vary widely between different T cell clones due to somatic gene re-arrangement and somatic hypermutation of T cell receptor genes (TCR alpha- and beta-genes). Individual T cell receptors can bind with low, intermediate or high affinity to complexes of major histocompatibility complex (MHC) molecules and short peptides bound to these MHC molecules. MHC molecules again fall into two relevant categories concerning the interaction between MHC molecules and T cells.

MHC class I molecules are composed of a heavy chain (the “alpha chain”) of approx. 43 kDa and a beta-2-microglobulin (“beta-2m”) with a molecular weight of approximately 12 kDa. The extracellular portion of the alpha-chain contains a domain capable of binding to short peptides with a length of 8 to 10 amino acid residues, if the peptide features amino acids with specific physico-chemical properties (bulkiness, hydrophobicity, electric charge, polarity, chirality) sufficiently matching the binding motif of the relevant peptide binding groove of the MHC class I molecule (Rammensee H G, Bachmann J, Emmerich N P N, Bachor O A, Stevanovic S. SYFPEITHI: database for MHC ligands and peptide motifs. Immunogenetics 1999; 50: 213-219. (www.syfpeithi.de); Rammensee H G, Bachmann J, Stevanovic S: MHC ligands and peptide motifs. Landes Bioscience 1997.).

MHC class II molecules are heterodimeric complexes consisting of an alpha-chain of 34 kD and a beta-chain of 29 kD. The alpha-chain and beta-chain together form with their alpha1 and beta1 domain a peptide binding cleft which can bind peptides with 9 or more amino acids. However, due to the specific enzymatic process of proteolytic cleavage of proteins taking place in the MHC class II-associated compartment, most peptide ligands have a length between 13 and 17 amino acid residues (see, for example, Chicz R M, Urban R G, Lane W S, Gorga J C, Stern L J, Vignali D A, Strominger J L. Predominant naturally processed peptides bound to HLA-DR1 are derived from MHC-related molecules and are heterogeneous in size. Nature, 1992; 358(6389):764-8).

Like with MHC class I molecules, the peptide binding groove of MHC class II molecules is selectively binding peptides with the appropriate length with sufficient affinity, if the amino acid sequence of the peptide features certain amino acids with specific physico-chemical properties in defined positions of the peptide sequence. The heavy chains of MHC class I molecules of humans are encoded by the HLA A, HLA B and HLA C genes of the HLA locus. The MHC class II alpha- and beta-chains of humans are encoded by the HLA DR, HLA DP and HLA DQ alpha and beta genes of the HLA locus. The HLA genes are highly polymorphic, giving rise to more than six hundred different HLA alleles for HLA class I alpha-chains and approximately five hundred HLA class II alpha- and beta-chains. The polymorphisms of the HLA genes influence the interaction between peptides and HLA alleles. For example, the HLA class I allele known as HLA-A*0201 prefers peptides with a leucine (L) or methionine (M) in position 2 from the N-terminal end of any given short peptide, and a valine (V) or leucine (L) in position 9 (the C-terminal end of a nonamer). In contrast, the HLA allele termed HLA B*1501 prefers binding peptides with a glutamine (Q) or leucine in position 2 and a phenylalanin (F) or tyrosine (Y) in position 9. Stable complexes between peptides and HLA alleles serve as specific interaction partners of specific TCRs.

Once ternary complexes are formed between HLA/peptide complexes on a living cell (the “target cell”) and a T cell carrying a matching TCR, the cell-cell interaction can trigger specific signal cascades leading to effects on both the cellular and molecular level of the T cell. Specifically, in the case of an interaction happening between the specific TCR of an activated CD8+ killer T cell (the “effector T cell”) and a HLA-positive human cell displaying a sufficient number of the matching HLA/peptide complex on its cell surface, a cytotoxic reaction of the T cell can be triggered. Such cytotoxic reactions of effector T cells lead to the destruction of the recognized target cell by osmotic lysis, which is induced by a perforation of the target cell membrane by perforin molecules excreted by the effector T cell. In this fashion, effector T cells are able to specifically destroy target cells displaying peptides in the context of MHC molecules which arise from intracellular pathogens (e.g., Influenza virus, Hepatitis C virus) or from self antigens (gene products encoded and expressed by the mammalian target cell itself). Self antigens that can be recognized by effector T cells in this manner include peptides with the appropriate length and physico-chemical properties from endogenous tumor-associated antigens, e.g., from MAGE gene products, p53, mdm-2, ras oncogene or Mucin-1 (for a summary, see, for example, www.cancerimmunity.org/peptidedatabase).

Cancer immunotherapy aims at using antigens that are, at best, exclusively expressed or over-expressed in tumor cells, as targets for therapy. Only a few tumor-associated antigens (TAAs) are expressed on the surface, e.g., HER-2/neu (Coussens L, Yang-Feng T L, Liao Y C, Chen E, Gray A, McGrath J, Seeburg P H, Libermann T A, Schlessinger J, Francke U (1985) Tyrosine kinase receptor with extensive homology to EGF receptor shares chromosomal location with neu oncogene. Science 230:1132-1139) or MUC-1 (Hayes D F, Mesa-Tejada R, Papsidero L D, Croghan G A, Korzun A H, Norton L, Wood W, Strauchen J A, Grimes M, Weiss R B (1991) Prediction of prognosis in primary breast cancer by detection of a high molecular weight mucin-like antigen using monoclonal antibodies DF3, F36/22, and CU18:a Cancer and Leukemia Group B study. J Clin Oncol 9:1113-1123), and may thus be targets for antibodies. Antibody-mediated therapy is also called “passive” immunotherapy because no other component of the patient's immune system requires specific activation. So far, antibody-mediated immunity has been used very successfully for preventive vaccination against infectious diseases and was the first form of immunotherapy to enter the market for therapeutic cancer treatment—e.g., with antibodies directed to HER-2/neu found in a fraction of mammary carcinomas (Leyland-Jones B (2002) Trastuzumab: hopes and realities. Lancet Oncol 3:137-144). However, most antigens are expressed in the cytosol or organelles of the cells and thus cannot be accessed by antibodies.

The recognition of target cells of the human body (or the body of any other mammal) displaying a naturally processed peptide ligand that has a functional counterpart on a T cell (a “T cell epitope”) requires that the T cell is activated prior to arousal from its naive state, a process called priming, which is assumed to be facilitated by so-called professional antigen-presenting cells (APCs). Dendritic cells (DCs) (Banchereau J, Paczesny S, Blanco P, Bennett L, Pascual V, Fay J, Palucka A K (2003) Dendritic cells: controllers of the immune system and a new promise for immunotherapy. Ann N Y Acad Sci 987:180-187) are considered the most prominent professional APCs, as they not only process antigens and present epitopes very well, but also—in their mature state—they bear high levels of co-stimulatory molecules on their surface, thereby providing the second signal required for a naive CD8-positive T cell to be transformed into a fully functional effector T cell. Because tumors usually cannot provide these co-stimulatory signals, the adaptive immune system ignores the tumor cells or, even worse, becomes tolerant toward cancer cells, falling into a state of anergy. The aim of peptide-based immunotherapy is to provide synthetic peptides from immunogenic tumor-associated antigens and to deliver these in a setting where effective priming of naive T cells can be accomplished.

Peptide-based immunization has many advantages over other modes of antigen delivery (e.g., proteins, viral vectors, or DNA vaccination): (1) Peptides are produced easily and rather inexpensively in clinical grade (GMP) quality; (2) Peptides have been proven safe and easy to administer in clinical settings; (3) Not only can they be used for vaccination, they are also appropriate for monitoring of specific immune responses using various in vitro and ex vivo T-cell assays.

Suematsu and Watanabe (Suematsu S, Watanabe T. Generation of a synthetic lymphoid tissue-like organoid in mice. Nat Biotechnol. 2004 December; 22(12):1539-45. Epub 2004 Nov. 28.) describe a tissue-engineered, lymphoid tissue-like organoid, which was constructed by transplantation of stromal cells embedded in biocompatible scaffolds into the renal subcapsular space in mice, and had an organized tissue structure similar to secondary lymphoid organs. This organoid contained compartmentalized B-cell and T-cell clusters. Furthermore, the organoid was transplantable to naive normal or severe combined immunodeficiency (SCID) mice, and antigen-specific, IgG-isotype antibody formation could be induced soon after intravenous administration of the antigen. The system is described as having possible applications in the treatment of immune deficiency.

Application of peptides capable of binding to HLA molecules has been shown to be very useful for priming naive T cells, transforming them into cytotoxic T cells with specificity for tumor cells owned to the tumor-associated antigens and peptides naturally processed from these tumor antigens. In a therapeutic setting, tumor-associated peptides can be applied in various ways. They can be either loaded in vitro onto the HLA molecules of mature dendritic cells prepared from peripheral blood monocytes of patient blood; this can be done, e.g., by culturing the PBMC with GM-CSF, IL-4, and TNF-alpha; after successful pulsing, the peptide-loaded DCs are usually injected subcutaneously, into the lymph nodes (intra-nodally), or intra-dermally. Alternatively and more elegantly, peptides can be administered directly into the dermis, where the Langerhans cells, a class of dendritic cells, reside. The latter way of administration does not require the tedious preparation of DCs in vitro; instead an effective adjuvant, which enhances the immunogenic effect of the peptides, is needed.

### OBJECT OF THE PRESENT INVENTION

The aim of peptide-based immunotherapy is to provide synthetic peptides from immunogenic tumor-associated antigens and to deliver these in a setting where effective priming of naive T cells can be accomplished. The administration of these peptides does require an effective adjuvant, which enhances the immunogenic effect of these peptides. Furthermore, improved methods of treatment for diseases that are related with TAAs are sought for. The present invention fulfils these needs. For the purposes of the present invention, all references as cited herein are incorporated by reference in their entireties.

### SUMMARY OF THE INVENTION

A novel method of treatment other than the aforementioned procedures and useful for eliciting strong T cell responses against defined peptides bound to MHC molecules on cell surfaces is described herein. This novel method of treatment is based on the use of implantable polyvinyl-alcohol (PVA) foams, which serve as an artificial lymph node. As will be described in further detail, surgically implanted PVA foams are rapidly infiltrated by various cellular components of the immune system. At the same time, these subcutaneous PVA foams are suitable for injection of peptides into the foam. It is also shown that this way of providing the therapeutic agent is superior to any conventional way of vaccination of mammals with peptides for the purpose of eliciting peptide-specific T cell immune responses.

The present invention, in a preferred aspect thereof, is directed to a method for producing a pharmaceutical composition for the improved activation of cells of the immune system, comprising the steps of: a) providing a biocompatible implantable foam material, and b) administering an effective amount of an active pharmaceutical ingredient into said foam, wherein said composition specifically activates cells of the immune system.

Preferably, said active pharmaceutical ingredient is selected from the group of proteins, peptides, and/or nucleic acids, more preferably derived from pathogens or endogenous antigens. In another preferred embodiment of the method according to the present invention, said endogenous antigens are wild-type or mutated antigens. Preferably, said endogenous antigens are selected from tumor-associated antigens. More preferably, said endogenous antigens are peptides being able to bind to MHC molecules, said MHC molecules being class I or class II molecules.

In another preferred embodiment of the method according to the present invention, said cells are selected from T cells, infiltrating T cells, B cells, NK cells, macrophages and dendritic cells, preferably selected from CD8 and CD4 positive cells. Most preferably, said CD4 and CD8 T cells are effector cells that can be hallmarked by an expression of, e.g., CD45R0, CCR7 and/or secretion of IFN-gamma and/or of Interleukin-2 and/or of Perforin.

In yet another preferred embodiment of the method according to the present invention, said peptide is administered in combination with one or more adjuvants. Preferably, said adjuvants are selected from oil-in-water emulsions, water-in-oil emulsions, ligands of toll-like receptors (TLR), and cytokines. The cytokines can be selected from the group of GM-CSF, Interleukin-2, Interleukin-7, Interleukin-12, Interleukin-15 and TNF-alpha, and said TLR ligands can be selected from synthetic or natural ligands, such as, e.g., nucleic acids (CpG oligonucleotides, RNA), Imiquimod (Aldara), lipopolysaccharides, and Gp96. A comprehensive listing of natural and synthetic TLR ligands can be found in Akira S, Takeda K, Nature Reviews Immunology, 2004; 4: 499-511.

In yet another preferred embodiment of the method according to the present invention, said foam material is selected from biocompatible polymers, such as PVA.

In yet another preferred embodiment of the method according to the present invention, said foam material is implanted into a subject before step b) as above. Preferably said implantation is subcutaneous (s.c.). In yet another preferred embodiment of the method according to the present invention, said subject is to be treated and is preferably a mammal, more preferably homo sapiens.

Another aspect of the present invention relates to pharmaceutical compositions that can be produced and are obtainable according to the method as above. Preferably, said pharmaceutical compositions are present in the form of an artificial lymph node, wherein said artificial lymph node functions as a reservoir for cells of the immune system.

Another aspect of the present invention then relates to a therapeutic kit, comprising, in identical or separate compartments thereof, a biocompatible implantable foam material as described above, and an active pharmaceutical ingredient as described above.

Another important aspect of the present invention relates to a method for improved immunotherapy, comprising the steps of: a) providing a biocompatible foam material, and b) administering an effective amount of a active pharmaceutical ingredient into said foam, whereby infiltrating T cells are specifically activated by said peptide.

Preferably, said active pharmaceutical ingredient is selected from the group of proteins, peptides, and/or nucleic acids, more preferably derived from pathogens or endogenous antigens. In another preferred embodiment of the method according to the present invention, said endogenous antigens are wild-type or mutated antigens. Preferably, said endogenous antigens are selected from tumor-associated antigens. More preferably, said endogenous antigens are peptides being able to bind to MHC molecules, said MHC molecules being class I or class II molecules.

In another preferred embodiment of the method according to the present invention, said cells are selected from T cells, more specifically alpha-beta-T-cells and gamma-delta-T-cells, which can be tumor-infiltrating T cells, B cells, Natural Killer (NK) cells, basophil, neutrophil or eosinophil granulocytes, macrophages and dendritic cells. Most preferably, said CD4 and CD8 T cells are effector cells that can be hallmarked by an expression of CD45R0 and/or secretion of IFN-gamma. Other preferred embodiments of the method for improved immunotherapy according to the present invention are as described above for the first aspect of the present invention.

In yet another preferred embodiment of the method according to the present invention, said immunotherapy is cancer immunotherapy. In yet another preferred embodiment of the method according to the present invention, said treatment elicits an anti-tumor immune response. Preferably said immune response is an immune response against a tumor-associated antigen.

Another aspect of the present invention is directed to a method for treatment of cancerous diseases, comprising an improved immunotherapy as described above. Said cancerous disease to be treated can be selected from, but shall not be limited to, the group of, e.g., renal, colon, ovarian, stomach, breast, prostate, lung, pancreatic, skin, brain and esophageal cancer.

### BRIEF DESCRIPTION OF THE DRAWINGS AND THE SEQUENCE PROTOCOL

FIG. 1 shows the immune responses of s.c. injection of SIINFEKL (SEQ ID No. 1) into the nape of the neck with peptide alone. Neither ovalbumin-transfected E.G7 mouse tumor cells of the H2-b haplotype, nor untransfected H2-b positive EL-4 mouse tumor cells are recognized by T cells from spleen or lymph nodes of immunized mice. In the Figure, the E:T ratio indicated the titration of T cells (effector cells) against target cells. Specific lysis is indicated in percent above unspecific background.

FIG. 2 shows the negative control. A 51Cr release assay (Brunner K T, Mauel J, Cerottini J C, Chapuis B. Quantitative assay of the lytic action of immune lymphoid cells on 51-Cr-labelled allogeneic target cells in vitro; inhibition by isoantibody and by drugs. Immunology. 1968, (2):181-96) with T cells from spleen and lymph nodes of control mice of the same strain (C57BL/6) that had not been immunized was performed as in FIG. 1.

FIG. 3 shows the immune responses of s.c. injection of SIINFEKL (SEQ ID No. 1) into s.c. implanted PVA foams. The injection of the ovalbumin-derived SIINFEKL (SEQ ID No. 1) peptide into s.c. implanted PVA foams led to the generation of SIINFEKL/H2-Kb specific T cells. Such specific T cells could be obtained from both spleen and lymph nodes of immunized mice. Both T cells from spleen or lymph node were able to lyse ovalbumin-transfected E.G7 cells. T cells from spleen led to a maximum lysis above unspecific background of approximately 45%. The lysis of target cells was a near-linear function of the E:T ratio.

FIG. 4 shows an independent second experiment as in FIG. 3 using the peptide ASNENMETM (SEQ ID No. 2) with T cells from axillary and cervical lymph nodes as well. The maximum lysis above unspecific background was 37%.

SEQ ID No. 1 shows the ovalbumin-derived SIINFEKL peptide (amino acid 257-264).

SEQ ID No. 2 shows the peptide ASNENMETM, derived from influenza nucleoprotein (amino acid 366-374).

SEQ ID No. 3 shows the phosphothioate-stabilized CpG deoxyoligonucleotide 1668 (TCC ATG ACG TTC CTG ATG CT).

### DETAILED DESCRIPTION OF THE INVENTION

The following definitions are offered as aids to understanding the terminology of the disclosure, but are in no way intended to limit the scope of the words included therein. To the extent the definitions offered herein expand the ordinary understanding of the terms, the expanded definition is intended. In any other case the offered definition is intended to augment and not to limit the ordinary definitions and any definitions customary in the related arts.

In the context of the present invention, an “artificial lymph node” is an artificial structure implanted into the body of a subject to be treated, whose structural features lead to a rapid infiltration of said structure by various cellular components of the immune system. One example for such an artificial lymph node is the biocompatible implantable foam material of the present invention that contains an effective amount of an active pharmaceutical ingredient integrated (i.e. physically associated) into said foam.

In the context of the present invention, a “biocompatible material” will be understood as a physical material that does not cause damage or adversely affect biological function when exposed to the tissue of an organism.

In the context of the present invention, an “implantable material” will be understood as a material that meets the various criteria to comply with either U.S. Food and Drug Administration (FDA) regulations or the International Organization for Standardization (ISO) requirements in order to be deemed fit for their intended use. Cytotoxicity studies are considered relevant to prove that the implant device is safe/biocompatible with human tissue. In vitro biocompatibility studies, based on the International Organization for Standardization 10993: Biological Evaluation of Medical Devices, Part 5: Tests for Cytotoxicity: in vitro Methods guidelines, can be conducted on the present invention to determine the potential for cytotoxicity.

In the context of the present invention, a pharmaceutical ingredient in “active”, if infiltrating T cells are specifically activated by said pharmaceutical ingredient.

The present invention, in a preferred aspect thereof, is directed to a method for producing a pharmaceutical composition for the improved activation of cells of the immune system, comprising the steps of: a) providing a biocompatible implantable foam material, and b) administering an effective amount of an active pharmaceutical ingredient into said foam, wherein said composition specifically activates cells of the immune system.

Preferably, said active pharmaceutical ingredient is selected from the group of proteins, peptides (i.e. oligopeptides or polypeptides), and/or nucleic acids (i.e. oligonucleotides or polynucletides, consisting of RNA, DNA, PNA, cNA or mixtures thereof), and is more preferably derived from pathogens or endogenous antigens. In another preferred embodiment of the method according to the present invention, said endogenous antigens are wild-type or mutated antigens. Preferably, said endogenous antigens are selected from tumor-associated antigens (for a summary, see, for example, Novellino L, Castelli C, Parmiani G. A listing of human tumor antigens recognized by T cells: March 2004 update. Cancer Immunol Immunother. 2005 March; 54(3):187-207. Epub 2004 Aug. 07.). More preferably, said endogenous antigens are peptides being able to bind to MHC molecules, said MHC molecules being class I or class II molecules.

In another preferred embodiment of the method according to the present invention, said cells are selected from T cells, infiltrating T cells, B cells, NK cells, macrophages and dendritic cells, and are preferably selected from CD8 and CD4 positive cells. Most preferably, said CD4 and CD8 T cells are effector cells that can be hallmarked by an expression of CD45R0, CCR7 and/or secretion of IFN-gamma and/or the secretion of Interleukin-2 and/or the secretion of Perforin. Respective assays for determining an expression of CD45R0, CCR7 and/or secretion of IFN-gamma and/or the secretion of Interleukin-2 and/or the secretion of Perforin are, e.g., ELISPOT assays and are known to the person of skill in the art and can be derived from the literature.

In yet another preferred embodiment of the method according to the present invention, said peptide is administered in combination with one or more adjuvants. Preferably, said adjuvants are selected from oil-in-water emulsions, water-in-oil emulsions, ligands of toll-like receptors (TLR), alum, heat-killed bacteria, CpG oligonucleotides, methylated bovine serum albumin, silica, and cytokines. The cytokines can be selected from GM-CSF, and said TLR ligands can be selected from synthetic or natural ligands, such as CpG oligonucleotides, Imiquimod (Aldara), lipopolysaccharides, and Gp96.

In yet another preferred embodiment of the method according to the present invention, said foam material is selected from biocompatible polymers, such as PVA. Suitable foam materials are well known to the person of skill in the present field and are selected from, amongst others, Poly(vinyl alcohol) synthetic polymer foams (see, for example, Li R H, White M, Williams S, Hazlett T. Poly(vinyl alcohol) synthetic polymer foams as scaffolds for cell encapsulation. J Biomater Sci Polym Ed. 1998;9(3):239-58.), chitosan (see, for example, Zielinski B A, Aebischer P. Chitosan as a matrix for mammalian cell encapsulation. Biomaterials. 1994 October; 15(13):1049-56.), poly(lactide-co-glycolide) foam scaffolds, porous methacrylate scaffolds, and three-dimensional polyurethane scaffolds. Many biocompatible polymer scaffolds are furthermore known as matrixes for tissue engineering. Most preferred is

In yet another preferred embodiment of the method according to the present invention, said foam material is implanted into a subject before step b) as above. Preferably said implantation is subcutaneous (s.c.). In yet another preferred embodiment of the method according to the present invention, said subject is to be treated and is preferably a mammal (such as a mouse, rat, cat, dog, horse, sheep, donkey, cow, rabbit, goat, monkey), and, more preferably, a human (homo sapiens).

Another aspect of the present invention relates to pharmaceutical compositions that can be produced and are obtainable according to the method as above. Preferably, said pharmaceutical compositions are present in the form of an artificial lymph node as described above, wherein said artificial lymph node functions as a reservoir for cells of the immune system.

Another aspect of the present invention then relates to a therapeutical kit, comprising, in identical or separate compartments thereof, a biocompatible implantable foam material as described above, and an active pharmaceutical ingredient as described above. The kit can furthermore contain additional chemicals, buffers, material (syringes, etc.), and manuals for use.

Another important aspect of the present invention relates to a method for improved immunotherapy, comprising the steps of: a) providing a biocompatible foam material, and b) administering an effective amount of a active pharmaceutical ingredient into said foam, whereby infiltrating T cells are specifically activated by said pharmaceutical ingredient. Other preferred embodiments of the method for improved immunotherapy according to the present invention are as described above for the first aspect of the present invention.

In yet another preferred embodiment of the method according to the present invention, said foam material is selected from biocompatible polymers, such as PVA. Other materials are as described above.

In yet another preferred embodiment of the method according to the present invention, said foam material is implanted into a subject before step b) as above. Preferably said implantation is subcutaneous (s.c.). In yet another preferred embodiment of the method according to the present invention, said subject is to be treated and is preferably a mammal (such as a mouse, rat, cat, dog, horse, sheep, donkey, cow, rabbit, goat, monkey), and, more preferably, a human (homo sapiens).

In yet another preferred embodiment of the method according to the present invention, said immunotherapy is cancer immunotherapy. In yet another preferred embodiment of the method according to the present invention, said treatment elicits an anti-tumor immune response. Preferably said immune response is an immune response against a tumor-associated antigen.

Another aspect of the present invention is directed to a method for treatment of cancerous diseases, comprising an improved immunotherapy as described above. Said cancerous disease to be treated can be selected from the group of renal, colon, ovarian, stomach, breast, prostate, lung, pancreatic, skin, brain and esophageal cancer.

Implantable PVA foams are known in the state of the art for wound closure therapy. Wound infections affecting soft tissues and bones are a serious complication after injury and endanger the reconstructive process. The goal of various treatments is to accelerate wound cleansing and thus to support wound healing. In the recent past, a treatment of wound infections and chronic wounds called vacuum-assisted closure (VAC) has been introduced (Argenta L C, Morykwas M J. Vacuum-assisted closure: a new method for wound control and treatment: clinical experience. Ann Plast Surg 1997; 38:563-76. Morykwas M J, Argenta L C, Shelton-Brown E I, McGuirt W. Vacuum-assisted closure: a new method for wound control and treatment: animal studies and basic foundation. Ann Plast Surg 1997; 38:553-62.). The VAC system consists of a polyvinyl alcohol (PVA) foam with a pore-size of 0.7±1.5 mm connected to a Redon drainage and a vacuum bottle. For wound drainage, the PVA foam is fitted to size according to the patient's injury and is placed within the wound leading the Redon drainage externally. The wound is then closed and a continuous suction is established. Various case reports and clinical studies investigating the efficiency of VAC application to either chronic or acute wounds have shown successful wound healing for almost all patients treated. Different animal models have been used to study the mechanisms leading to the positive effect of VAC in wound healing. Experimentally induced wounds in pigs were treated with VAC and healing was compared to control wounds treated with saline-moistened gauze; the results showed an increase in local blood flow with an improved supply of nutrients, a decrease in bacterial count following removal of excess wound fluid and an accelerated rate of granulation tissue formation. A second reason for the positive effect of VAC may be the reduction of post-traumatic immuno-suppression mediated by the continuous drainage of fluid from the wound. It was shown by Gouttefangeas et al. (Gouttefangeas C, M. Eberle, P. Ruck, M. Stark, J. E. Mueller, H-D. Becker, H-G. Rammensee, J. Pinocy. Functional T lymphocytes infiltrate implanted polyvinyl alcohol foams during surgical wound closure therapy. Clin Exp Immunol 2001; 124: 398-405.) that the VAC system allows leukocytes to infiltrate and contact the wound, and so promotes an efficient immune reaction against the local infection. Moreover, the tight contact established by the Redon drainage system between the wound surface and the PVA foam even allows infiltration of immunocompetent cells, mainly granulocytes, but also CD41 and CD81 T cells, into the PVA foam itself, including T-, B-, Dendritic and NK-Cells. The CD4- and CD8-positive T cells found in the PVA foam are in an activated state (CD45RO⁺) and can be stimulated with common super antigens or recall antigens. From this perspective, PVA foams are similar to lymph nodes and thus represent a kind of artificial lymph nodes. As such, PVA foams represent an optimal environment for vaccination and induction of immune responses.

For the first time ever, implantable PVA foams were used as a reservoir for synthetic peptides suitable for specifically binding to MHC class I molecules of the host. Injecting peptide into the PVA foam leads to significant T cell responses against cells carrying the respective HLA class I molecule/peptide complexes on their surfaces. Specifically, 10 days after s.c. implantation of PVA foams, strong T cell responses from CD8+ T cells against, e.g., the model peptide ligand of the mouse MHC class I allele H2-Kb, SIINFEKL (SEQ ID No. xx) from chicken ovalbumin (Falk K, Rotzschke O, Stevanovic S, Jung G, Rammensee H G. Allele-specific motifs revealed by sequencing of self-peptides eluted from MHC molecules. Nature 1991; 351(6324): 290-296.), can be detected. Experiments leading to the invention were conducted as described in the following section of this document.

The invention shall now be further described in the following examples that are related to preferred embodiments of the present invention, without being limited to these examples.

Materials and Animals:

V.A.C® Soft-Foams (10×15 cm, product no. M6275034, KCI Medical Ltd. Wimborne, UK) were used for implantation. Female mice (strain C57BL/6, age: 8-10 weeks, MHC haplotype H-2b) were obtained from Charles River Laboratories (Wilmington, Mass., USA) and maintained in the animal facilities of the Department of Immunology, Institute for Cell Biology, University of Tubingen, Germany. Animal health was supervised by the veterinary department of the University of Tubingen. For foam soaking, a solution of 20 μg/ml gentamicin in Modified Eagle's medium (alpha-modification; aMEM) was prepared (both Sigma-Aldrich, Deisenhofen, Germany).

Analgesia was performed with Rimadyl® (Pfizer GmbH, Karlsruhe). The stock solution of 5 mg/ml was diluted immediately before usage with sterile PBS to obtain a working solution of 100 μg/ml. For anesthesia, a mixture of 5 μg/ml Fentanyl (ratio-pharm GmbH, Ulm, Germany), 0.5 mg/ml Dormicum® (Roche Pharma AG, Rheinach, Swizerland), and 50 μg/ml Dormitor® (Pfizer GmbH) was used. The antidote mixture contained 120 μg/ml Naloxon (ratiopharm GmbH), 50 μg/ml Anexate® (Roche Pharma AG) and 250 μg/ml Antisedan (Pfizer GmbH). All drugs were obtained from the veterinary department of the University of Tubingen except Vidisic® eye gel (Dr. Mann Pharma, Berlin, Germany) which was purchased from local pharmacies. Wound closure was performed using PDS II suture (Ethicon GmbH, Norderstedt, Germany) and Flint wound spray dressing (Togal-Werk, Munich, GmbH).

Peptides having the sequence SIINFEKL (SEQ ID No. 1), derived from chicken ovalbumin (amino acid 257-264) and ASNENMETM (SEQ ID No. 2), derived from influenza nucleoprotein (amino acid 366-374) were synthesized and tested using mass spectrometry by the peptide chemistry facilities in the Department of Immunology (University of Tübingen, Tübingen, Germany). Titermax classic was obtained from Sigma-Aldrich, phosphothioate-stabilised CpG deoxyoligonucleotide 1668 (TCC ATG ACG TTC CTG ATG CT (SEQ ID No. 3)) was purchased from TIB MOLBIOL (Berlin, Germany). Mouse granulocyte-macrophage colony-stimulating factor (GM-CSF) was obtained from Biosource Int. (Camarillo, Calif., USA).

Preparation of Foam Material Prior to Implantation:

From bulk Poly(vinyl alcohol) (PVA) foam material, pieces with a size of 1 cm×0.5 cm×0.5 cm were prepared from bulk PVA foam material under sterile conditions in a petridish. These pieces of PVA foam were the soaked and stored in aMEM cell culture medium containing Gentamicin in a final concentration of 20 μg/ml (1:500 dilution of stock material) in petridishes under sterile conditions.

Surgical Implantation of PVA Foams

Analgesia: 100 μl of working solution of Rimadyl was injected subcutaneously into the nape of the neck 3 hours prior to surgical implantation of PVA foams.

Anesthesia: 100 μl of the narcotic mixture per 10 g of mouse body weight were injected intraperitoneally corresponding to a dose of 50 μg/kg body weight Fentanyl, 5 mg/kg body weight Dormicum®, and 0.5 μg/kg body weight Dormitor®. The mice were observed for 5 minutes after injection and further 50 μl were injected only, if the animals were not sufficiently anaesthetized. Anaesthetized animals were protected against hypothermia, Vidisic® gel was applied on the corneas of the eyes to protect them against drying out. Then, the skin on the back of the animal next to the tail was clean-shaven.

Surgery: An incision with a length of 1 cm was made on the clean-shaven part of the back next to the tail. A subcutaneous pocket on both sides of the dorsal thoracic wall was prepared and, immediately after, two pieces of PVA foam soaked in aMEM were implanted. The incision was closed by sewing a continuous suture and subsequent application of a spray bandage. Finally, 100 μl of antidote mixture per 10 g of mouse body weight antidote mixture was applied intraperitoneally corresponding to 1.2 mg/kg body weight Naloxon, 0.5 mg/kg body weight Anexate®, and 2.5 mg/kg body weight Antisedan®. Animals were observed until recovery from anesthesia.

Follow-up care: On day 2 after surgery, the analgetic Rimadyl was applied as described above. In rare cases of dehiscence of the suture, a veterinarian of the University of Tübingen was consulted and the wound was treated with a zinc-containing ointment and/or recovered with a wound spray (Flint wound spray dressing, Togal-Werk, Munich, GmbH).

Immunization: On day 9 after surgery, wounds had generally healed and healthy mice were used for immunization with 30 μg peptide per mouse. Depending on the experiment, CpG deoxyoligonucleotide 1668 (5 -100 pmole/mouse), mouse GM-CSF (10³ units/mouse) and Titermax classic were used as adjuvants. Generally, injection volume was adjusted to 200 μl per mouse with sterile PBS. When applying Titermax classic, all other injection components were mixed and then added in small portions to an equal volume of Titermax classic. After each addition, the vial was rigorously mixed to obtain a white, semi-fluid water-in-oil suspension. Injection was either performed subcutaneously into the nape of the neck, the base of tail and the footpad, using a volume ratio of 3:1:1, respectively, or directly into the centre of the right foam implantate.

T-cell culture and ⁵¹Cr release assay: On day 9 after immunization, mice were sacrificed and 2*10⁷ splenocytes were stimulated in vitro for 5 days using 10⁷ irradiated (30 Gy) splenocytes from untreated C57BL/6 mice as feeder cells and 5 ng/ml peptide. Cytotoxic T lymphocyte (CTL) activity was measured in a standard ⁵¹Cr release assay (Brunner et al., 1968). Briefly, stimulated cells were harvested, counted and plated out in a series of 51:3-dilutions in a round-bottom 96-well plate. 10⁶ EL-4 cells (C57BL/6-derived T cell lymphoma; ATCC no. TIB-39) were loaded with 50 pmole peptide and [⁵¹Cr] Na₂CrO₄-labeled at 37° C. for 30 min. After intense washing with cell culture medium, 10⁴ of these cells were added to each well of the 96-well plate as targets. ⁵¹Cr release was measured after 4 h incubation at 37° C./5% (v/v) CO₂.

Results

The peptide under investigation for demonstrating the proposed utility of PVA foams as artificial lymph nodes was SIINFEKL (SEQ ID No. 1) from chicken ovalbumin.

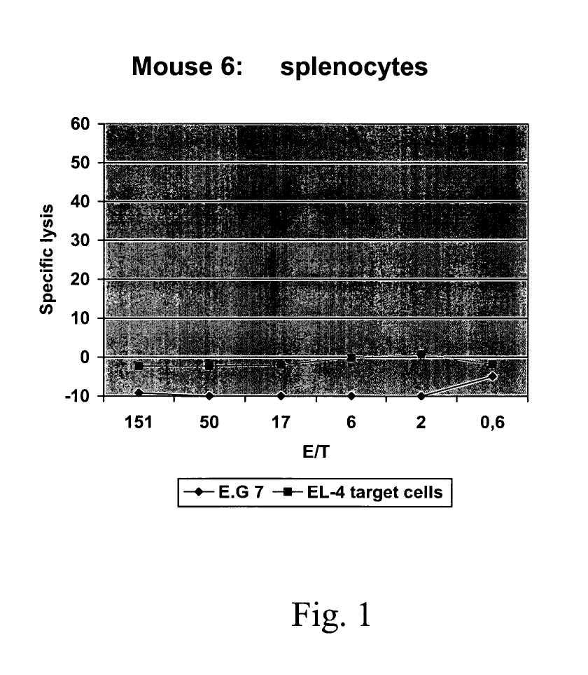

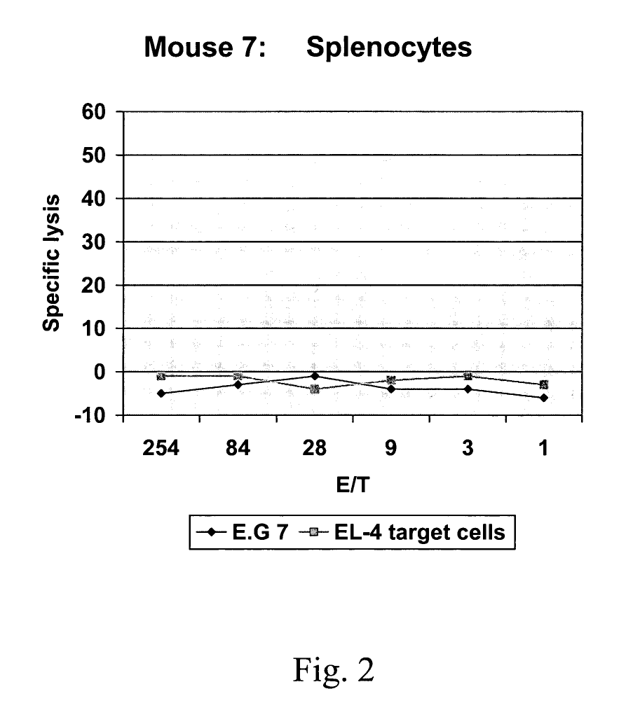

a) Subcutaneous (s.c.) immunization with peptide alone: The s.c. injection of SIINFEKL (SEQ ID No. 1) into the nape of the neck did not lead to detectable immune responses. Neither ovalbumin-transfected E.G7 mouse tumor cells of the H2-b haplotype, nor untransfected H2-b positive EL-4 mouse tumor cells were recognized by T cells from spleen or lymph nodes of immunized mice (FIG. 1). In the Figures, the E:T ratio indicated the titration of T cells (effector cells) against target cells. Specific lysis is indicated in percent above unspecific background. As a negative control, a 51Cr release assay with T cells from spleen and lymph nodes of control mice of the same strain (C57BL/6) that had not been immunized was performed. No measurable immune response against target cells was detected (FIG. 2).

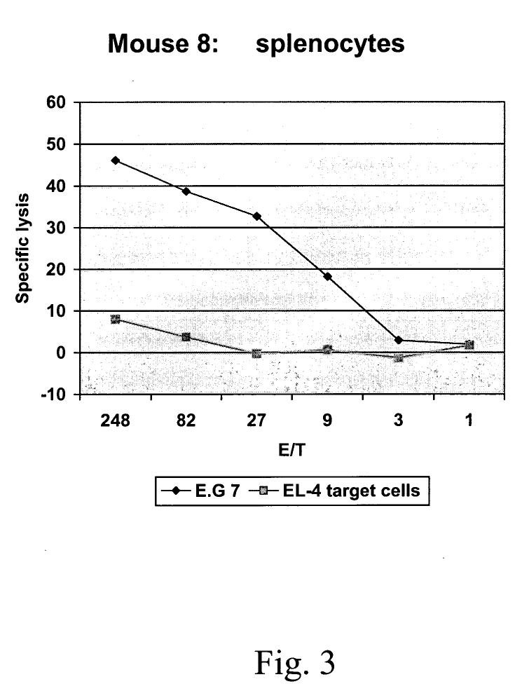

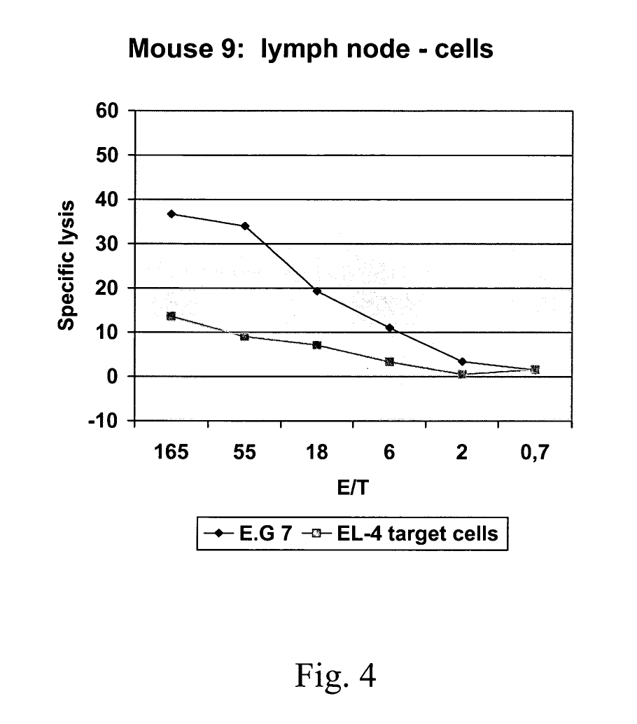

b) Immunization with peptide SIINFEKL into s.c. implanted PVA foams: The injection of the ovalbumin-derived SIINFEKL (SEQ ID No. 1) peptide into s.c. implanted PVA foams led to the generation of SIINFEKL/H2-Kb specific T cells. Such specific T cells could be obtained from both spleen and lymph nodes of immunized mice. Both T cells from spleen or lymph node were able to lyse ovalbumin-transfected E.G7 cells. T cells from spleen led to a maximum lysis above unspecific background of approximately 45%. The lysis of target cells was a near-linear function of the E:T ratio (FIG. 3). In an independent second experiment, the results could be repeated with the peptide ASNENMETM (SEQ ID No. 2) and T cells from axillary and cervical lymph nodes as well. The maximum lysis above unspecific background was 37% (FIG. 4).

Conclusion: The implantation of PVA foams and subsequent administration of peptide into the foam leads to specific activation of infiltrating T cells. These T cells can obviously function as effector T cells with the ability to recognize and lyse target cells displaying the peptide, which can be loaded externally or which can be processed from endogenously expressed antigen, in the context of the appropriate MHC molecule. This method allows for a surprisingly more effective treatment that can be employed in immunotherapy of diseases, specifically peptide-based cancer immunotherapy.

What is claimed is:

1. A method for producing a pharmaceutical composition for the improved activation of cells of the immune system, comprising the steps of:
a) providing a biocompatible implantable foam material, and
b) administering an effective amount of an active pharmaceutical ingredient into said foam,
wherein said composition specifically activates cells of the immune system.

2. The method according to claim 1, wherein said active pharmaceutical ingredient is selected from the group consisting of proteins, peptides and nucleic acids.

3. The method according to claim 2, wherein said ingredient is derived from a pathogen or endogenous antigen.

4. The method according to claim 3, wherein said endogenous antigen is a wild-type or mutated antigen.

5. The method according to claim 3, wherein said endogenous antigen is selected from tumour-associated antigens.

6. The method according to claim 3, wherein said endogenous antigen is a peptide that is able to bind to a MHC molecules.

7. The method according to claim 6, wherein said MHC molecule is a class I or class II molecule.

8. The method according to claim 1, wherein said cells are selected from the group consisting of alpha-beta T cells, gamma-delta T-cells, B cells, NK cells, macrophages, baso-, neutro- or eosinophilic granulocytes and dendritic cells.

9. The method according to claim 8, wherein said T cells are selected from CD8 and CD4 positive cells.

10. The method according to claim 9, wherein said CD4 and CD8 T cells are effector cells.

11. The method according to claim 10, wherein said effector cells are hallmarked by at least one characteristic selected from the group consisting of expression of CD45R0 or CCR7; secretion of IFN-gamma; secretion of Interleukin-2; and secretion of Perforin.

12. The method according to claim 2, wherein said peptide is administered in combination with one or more adjuvants.

13. The method according to claim 12, wherein said adjuvant is selected from the group consisting of oil-in-water emulsions, water-in-oil emulsions, ligands of toll-like receptors (TLR), alum, heat-killed bacteria, CpG oligonucleotides, methylated bovine serum albumin, silica, and cytokines.

14. The method according to claim 13, wherein said cytokine is selected from the group consisting of GM-CSF, IL-2, IL-7, IL-12, IL-15 and TNF-alpha.

15. The method according to claim 13, wherein said TLR ligand is selected from the group consisting of CpG oligonucleotides, Imiquimod (Aldara), lipopolysaccharides and Gp96.

16. The method according to claim 1, wherein said foam material is a biocompatible polymer.

17. The method according to claim 1, wherein said foam material is implanted into a subject before step b).

18. The method according to claim 17, wherein said implantation is subcutaneous.

19. The method according to claim 17, wherein said subject is a mammal.

20. The method according to claim 19, wherein said subject is a human.

21. A pharmaceutical composition for the improved activation of cells of the immune system, wherein said composition is obtainable by a method comprising the steps of:
a) providing a biocompatible implantable foam material, and
b) administering an effective amount of an active pharmaceutical ingredient into said foam,
wherein said composition specifically activates cells of the immune system.

22. The pharmaceutical composition, according to claim 21, wherein said foam material is implanted into a subject before step b).

23. The pharmaceutical composition, according to claim 22, in the form of an artificial lymph node, wherein said artificial lymph node functions as a reservoir for cells of the immune system.

24. A therapeutical kit comprising, in one or more compartments thereof, a biocompatible implantable foam material, and an active pharmaceutical ingredient, wherein when said pharmaceutical ingredient is administered into said foam a composition is produced that specifically activates cells of the immune system.

25. A method for improved immunotherapy, comprising the steps of:
a) providing a biocompatible foam material, and
b) administering an effective amount of a active pharmaceutical ingredient into said foam,
whereby infiltrating immune system cells are specifically activated by said foam comprising said pharmaceutical ingredient.

26. The method according to claim 25, wherein said active pharmaceutical ingredient is selected from the group consisting of proteins, peptides and nucleic acids.

27. The method according to claim 26, wherein said ingredient is derived from a pathogen or endogenous antigen.

28. The method according to claim 27, wherein said endogenous antigen is a wild-type or mutated antigen.

29. The method according to claim 27, wherein said endogenous antigen is selected from tumor-associated antigens.

30. The method according to claim 27, wherein said endogenous antigen is a peptide that is able to bind to a MHC molecule.

31. The method according to claim 30, wherein said MHC molecule is a class I or class II molecule.

32. The method according to claim 25, wherein said cells are selected from the group consisting of T cells, infiltrating T cells, B cells, NK cells, macrophages and dendritic cells.

33. The method according to claim 32, wherein said T cells are selected from CD8 and CD4 positive cells.

34. The method according to claim 33, wherein said CD4 and CD8 T cells are effector cells.

35. The method according to claim 34, wherein said effector cells are hallmarked by having at least one characteristic selected from the group consisting of expression of CD45R0 and secretion of IFN-gamma.

36. The method according to claim 26, wherein said peptide is administered in combination with one or more adjuvants.

37. The method according to claim 36, wherein said adjuvant is selected from the group consisting of oil-in-water emulsions, water-in-oil emulsions, ligands of toll-like receptors (TLR), alum, heat-killed bacteria, CpG oligonucleotides, methylated bovine serum albumin, silica and cytokines.

38. The method according to claim 37, wherein said cytokine is selected from the group consisting of GM-CSF, IL-2, IL-7, IL-1 2, IL-15 and TNF-alpha.

39. The method according to claim 37, wherein said TLR ligand is selected from the group consisting of CpG oligonucleotides, Imiquimod (Aldara), lipopolysaccharides and Gp96.

40. The method according to claim 25, wherein said foam material is a biocompatible polymer.

41. The method according to claim 25, wherein said foam material is implanted into a subject before step b).

42. The method according to claim 41, wherein said implantation is subcutaneous.

43. The method according to claim 41, wherein said subject is a mammal.

44. The method according to claim 41, wherein said subject is a human.

45. The method according to claim 25, wherein said immunotherapy is cancer immunotherapy.

46. The method according to claim 25, wherein said treatment elicits an anti-tumor immune response

47. The method according to claim 46, wherein said immune response is an immune response against a tumor-associated antigen.

48. The method, according to claim 25, used to treat a cancerous disease.

49. The method, according to claim 48, wherein said cancerous disease is selected from the group consisting of renal, colon, ovarian, stomach, breast, prostate, lung, pancreatic, skin, brain and esophageal cancer.


SEQUENCE LISTING (3 sequences)

SEQ ID NO: 1 - protein, 8 aa, Artificial Sequence
  Ovalbumin-derived SIINFEKL peptide (amino acid 257-264)
SIINFEKL

SEQ ID NO: 2 - protein, 9 aa, Artificial Sequence
  peptide ASNENMETM derived from influenza nucleoprotein (amino acid 366-374)
ASNENMETM

SEQ ID NO: 3 - DNA, 20 bp, Artificial Sequence
  phosphothioate-stabilized CpG deoxyoligonucleotide 1668
tccatgacgttcctgatgct